(19) United States
(12) Patent Application Publication
(10) Pub. No.: US 2025/0249236 A1
(43) Pub. Date: Aug. 7, 2025

(54) ELECTRICAL STIMULATION CUFF LEADS WITH CONJOINED DISTAL END PORTIONS

(71) Applicant: Boston Scientific Neuromodulation Corporation, Valencia, CA (US)

(72) Inventors: Ranjan Krishna Mukhari Nageri, Valencia, CA (US); Ryan Michael Baca, Arleta, CA (US); Hari Hara Subramanian, Valencia, CA (US); Jonathan Daniel Walden, Moorpark, CA (US)

(21) Appl. No.: 19/043,997

(22) Filed: Feb. 3, 2025

Related U.S. Application Data
(60) Provisional application No. 63/549,797, filed on Feb. 5, 2024.

(51) Int. Cl. A61N 1/05; A61N 1/36

(52) U.S. Cl. CPC A61N 1/0556; A61N 1/3605

(57) ABSTRACT

A cuff lead arrangement includes a first cuff lead having a first cuff body; first stimulation emitters (for example, electrodes) disposed on an interior surface of the first cuff body; a first lead body coupled to the first cuff body; first conductors electrically coupled to the first stimulation emitters and extending along the first lead body; and at least one first connection element. The cuff lead arrangement further includes a second cuff lead having a second cuff body; second stimulation emitters disposed on an interior surface of the second cuff body; a second lead body coupled to the second cuff body; second conductors electrically coupled to the second stimulation emitters and extending along the second lead body; and at least one second female connection element configured to receive the at least one first male connection element to conjoin the first cuff lead to the second cuff lead.

### CROSS-REFERENCE TO RELATED APPLICATIONS

This application claims the benefit under 35 U.S.C. § 119 (e) of U.S. Provisional Patent Application Ser. No. 63/549,797, filed Feb. 5, 2024, which is incorporated herein by reference.

### FIELD

The present disclosure is directed to the area of implantable electrical stimulation systems and methods of making and using the systems. The present disclosure is also directed to implantable electrical stimulation cuff devices, as well as methods of making and using the same.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is a cuff lead arrangement for electrical stimulation of a nerve. The cuff lead arrangement includes a first cuff lead having a first cuff body configured to be disposed around at least a portion of the nerve, the first cuff body including an exterior surface, an interior surface, and at least one first male connection element; a plurality of first electrodes disposed on the interior surface of the first cuff body; a first lead body coupled to the first cuff body; and a plurality of first conductors electrically coupled to the first electrodes and extending along the first lead body. The cuff lead arrangement further includes a second cuff lead having a second cuff body configured to be disposed around at least a portion of the nerve, the second cuff body including an exterior surface, an interior surface, and at least one second female connection element configured to receive the at least one first male connection element to conjoin the first cuff body to the second cuff body; a plurality of second electrodes disposed on the interior surface of the second cuff body; a second lead body coupled to the second cuff body; and a plurality of second conductors electrically coupled to the second electrodes and extending along the second lead body.

In at least some aspects, each of the at least one first male connection element includes a connection finger and each of the at least one second female connection element includes a slot configured to receive a portion of the connection finger to conjoin the first cuff body to the second cuff body.

In at least some aspects the at least one first male connection element includes a first end portion of the first cuff body having a reduced outer diameter relative to a first central portion of the first cuff body and the at least one second female connection element includes a second end portion of the second cuff body having a larger inner diameter relative to a second central portion of the second cuff body, wherein the second end portion of the second cuff body is configured to receive therein the first end portion of the first cuff body to conjoin the first cuff body to the second cuff body.

In at least some aspects the first cuff body further includes a first strain relief extending along a portion of the first lead body wherein the first lead body is attached to the first cuff body, wherein the at least one male connection element is part of the first strain relief. In at least some aspects each of the at least one first male connection element includes a pin and each of the at least one second female connection element includes a slot configured to receive the pin to conjoin the first cuff body to the second cuff body.

In at least some aspects, the at least one first male element and the at least one second female connection element include at least one of: a bolt-and-hole arrangement, a nut-and-hole arrangement, a screw-and-hole arrangement, or a thread-and-elbow arrangement. In at least some aspects, the first cuff body further includes at least one first female connection element and the second cuff body further includes at least one second male connection element configured to receive the at least one first female connection element to conjoin the first cuff body to the second cuff body.

In at least some aspects, the first cuff body further includes a plurality of the first male connection elements and the second cuff body further includes a plurality of the second female connection elements configured to receive the first male connection elements to conjoin the first cuff body to the second cuff body.

In at least some aspects, the second cuff body further includes at least one second male connection element, the cuff lead arrangement further including a third cuff lead, including a third cuff body configured to be disposed around at least a portion of the nerve, the third cuff body including an exterior surface, an interior surface, and at least one third female connection element configured to receive the at least one second male connection element to conjoin the second cuff body to the third cuff body; a plurality of third electrodes disposed on the interior surface of the third cuff body; a third lead body coupled to the third cuff body; and a plurality of third conductors electrically coupled to the third electrodes and extending along the third lead body.

In at least some aspects, the first electrodes are disposed in a first arrangement on the interior surface of the first cuff lead and the second electrodes are disposed in the first arrangement on the interior surface of the second cuff lead. In at least some aspects, when conjoined, the second electrodes in the first arrangement are rotationally offset from the first electrodes in the first arrangement.

Another aspect is a cuff lead arrangement for electrical stimulation of a nerve. The cuff lead arrangement includes a first cuff lead having a first cuff body configured to be disposed around at least a portion of the nerve; a plurality of first electrodes disposed on an interior surface of the first cuff body; a first lead body including a first distal attachment portion directly attached to the first cuff body; a plurality of first conductors electrically coupled to the first electrodes and extending along the first lead body; and at least one first connection element coupled to the first cuff body or the first distal attachment portion of the first lead body. The cuff lead arrangement also includes a second cuff lead having a second cuff body configured to be disposed around at least a portion of the nerve; a plurality of second electrodes disposed on an interior surface of the second cuff body; a second lead body including a second distal attachment portion directly attached to the second cuff body; a plurality of second conductors electrically coupled to the second electrodes and extending along the second lead body; and at least one second connection element coupled to the second cuff body or the second distal attachment portion of the second lead body, wherein the at least one second connection element is configured to be attached to the at least one first connection element to conjoin the first cuff lead to the second cuff lead.

In at least some aspects, the at least one first connection element and the at least one second connection element include at least one of: a tongue-and-groove arrangement, a screw arrangement, a bolt arrangement, a thread-and-elbow arrangement, a pin-and-slot arrangement, a connection-finger-and-slot arrangement, or a sliding arrangement. In at least some aspects, the at least one first connection element includes a first eyelet coupled to the first cuff body or the first distal attachment portion, the at least one second connection element includes a second eyelet coupled to the second cuff body or the second distal attachment portion, and the first eyelet and the second eyelet are configured to be sutured together.

In at least some aspects, the second cuff lead further includes at least one third connection element coupled to the second cuff body of the second distal attachment portion of the second lead body and the cuff lead arrangement further includes a third cuff lead having a third cuff body configured to be disposed around at least a portion of the nerve; a plurality of third electrodes disposed on an interior surface of the third cuff body; a third lead body including a third distal attachment portion directly attached to the third cuff body; a plurality of third conductors electrically coupled to the third electrodes and extending along the third lead body; and at least one third connection element coupled to the third cuff body or the third distal attachment portion of the third lead body, wherein the at least one third connection element is configured to be attached to the at least one second connection element to conjoin the second cuff lead to the third cuff lead.

Yet another aspect is an electrical stimulation system that includes any of the cuff lead arrangement described above and a control module configured to receive a portion of the first lead body and to electrically couple to the plurality of first electrodes and to receive a portion of the second lead body and to electrically couple to the plurality of second electrodes, wherein the control module is configured to generate electrical stimulation signals for delivery through at least one of the first or second electrodes.

A further aspect is a method of electrically stimulating a patient using an electrical stimulation system with any of the cuff lead arrangements described above. The method includes providing the cuff lead arrangement implanted within the patient; generating stimulation signals by the control module; and delivering the stimulation signals from the control module to the patient using at least one of the first or second electrodes.

Another aspect is a cuff lead arrangement for electrical stimulation of a nerve. The cuff lead arrangement includes a first cuff lead having a first cuff body configured to be disposed around at least a portion of the nerve; a plurality of first stimulation emitters disposed on the interior surface of the first cuff body; a first lead body including a first distal attachment portion directly attached to the first cuff body; a plurality of first conductors operatively coupled to the first stimulation emitters and extending along the first lead body; and at least one first connection element coupled to the first cuff body or the first distal attachment portion of the first lead body. The cuff lead arrangement also includes a second cuff lead having a second cuff body configured to be disposed around at least a portion of the nerve; a plurality of second stimulation emitters disposed on the interior surface of the second cuff body; a second lead body including a second distal attachment portion directly attached to the second cuff body; a plurality of second conductors operatively coupled to the second stimulation emitters and extending along the second lead body; and at least one second connection element coupled to the second cuff body or the second distal attachment portion of the second lead body, wherein the at least one second connection element is configured to be attached to the at least one first connection element to conjoin the first cuff lead to the second cuff lead.

Yet another aspect is a stimulation system that includes any of the cuff lead arrangements described above and a control module configured to receive a portion of the first lead body and to couple to the plurality of first stimulation emitters (e.g., first electrodes) and to receive a portion of the second lead body and to couple to the plurality of second stimulation emitters (e.g., second electrodes), wherein the control module is configured to generate stimulation signals for delivery through at least one of the first or second stimulation emitters.

A further aspect is a method of stimulating a patient using a stimulation system with any of the cuff lead arrangements described above. The method includes providing the cuff lead arrangement implanted within the patient; generating stimulation signals by the control module; and delivering the stimulation signals from the control module to the patient using at least one of the first or second stimulation emitters (e.g., first or second electrodes).

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1A is a schematic view of one embodiment of an electrical stimulation system that includes a cuff lead electrically coupled to a control module;

FIG. 1B is a schematic view of one embodiment of a portion of another cuff lead for use with the electrical simulation system of FIG. 1A;

FIG. 1C is a schematic view of an electrical stimulation system of FIG. 1A that includes a splitter with the cuff lead;

FIG. 2A is a schematic view of one embodiment of the control module of FIG. 1A configured and arranged to electrically couple to at least one elongated device;

FIG. 2B is a schematic view of one embodiment of a lead extension configured and arranged to electrically couple the elongated device of FIG. 2A to the control module of FIG. 1A;

FIG. 3A is a schematic perspective view of one embodiment of a distal end portion of a cuff lead that is conjoinable with the distal end portion of another cuff lead;

FIG. 3B is a schematic view of one embodiment of a cuff body of a first cuff lead conjoined with a cuff body of a second cuff lead;

FIG. 4A is a schematic cross-sectional view of one embodiment of a lead clip for conjoining two cuff leads;

FIG. 4B is a schematic cross-sectional view of one embodiment of portions of two cuff leads that can be conjoined by sliding the end portion of one cuff lead over the end portion of another cuff lead;

FIG. 4C is a schematic cross-sectional view of one embodiment of portions of two cuff leads that can be conjoined using a pin-and-slot arrangement;

FIG. 5 is a cross-sectional view of one embodiment of cuff disposed around a portion of a nerve;

FIG. 6A is a plan view of one embodiment of two cuff bodies having aligned electrode arrangements;

FIG. 6B is a plan view of one embodiment of two cuff bodies having offset or staggered electrode arrangements;

FIG. 7 is a schematic side view of one embodiment of conjoined cuff bodies; and

FIG. 8 is a schematic block diagram of one embodiment of an electrical stimulation arrangement.

### DETAILED DESCRIPTION

The present invention is directed to the area of implantable electrical stimulation systems and methods of making and using the systems. The present invention is also directed to implantable electrical stimulation cuff devices, as well as methods of making and using the same.

Suitable implantable electrical stimulation systems include, but are not limited to, at least one lead with one or more electrodes disposed along a distal end portion of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,203,548; 7,244,150; 7,450,997; 7,596,414; 7,610,103; 7,672,734; 7,761,165; 7,783,359; 7,792,590; 7,809,446; 7,949,395; 7,974,706; 6,175,710; 6,224,450; 6,271,094; 6,295,944; 6,364,278; 6,391,985; 7,596,414; 7,974,706; 8,831,742; 8,423,157; 10,485,969; 10,493,269; 10,709,888 and 10,485,969; U.S. Patent Application Publications Nos. 2007/0150036; 2008/0304830; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2017/0333692; 2018/0154156; 2022/0370793; 2022/0395690; and 2022/03770793; and U.S. Provisional Patent Application Ser. No. 63/522,367, all of which are incorporated by reference in their entireties.

Electrical stimulation systems are used in the description below, but it will be understood that the electrical stimulation system, electrical stimulation lead, and electrodes can be replaced, in whole or in part, by an optical stimulation system (or electrical/optical stimulation system), optical stimulation lead, and optical emitters to produce optical stimulation or photobiomodulation. Examples of optical stimulation systems and electrical/optical stimulation systems, which include one or more optical emitters instead, or in addition to electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties. Electrodes and optical emitters are examples of stimulation emitters.

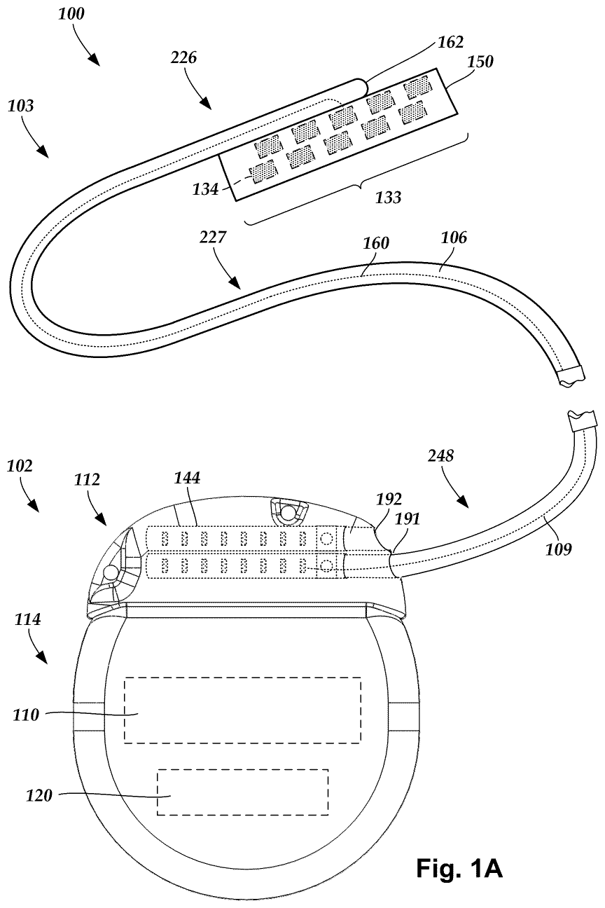

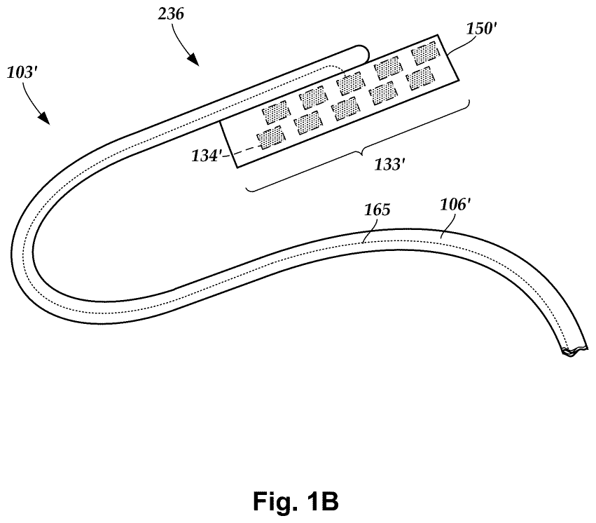

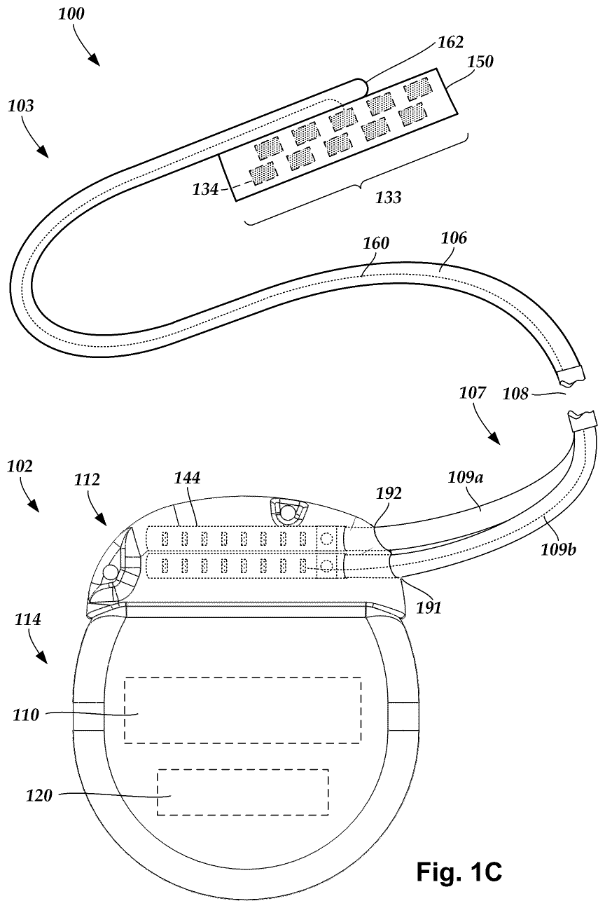

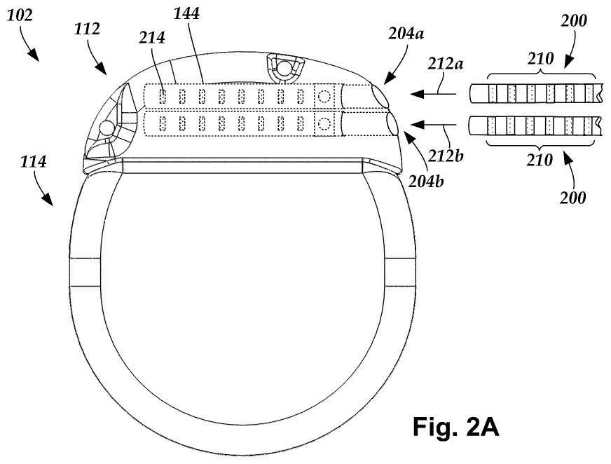

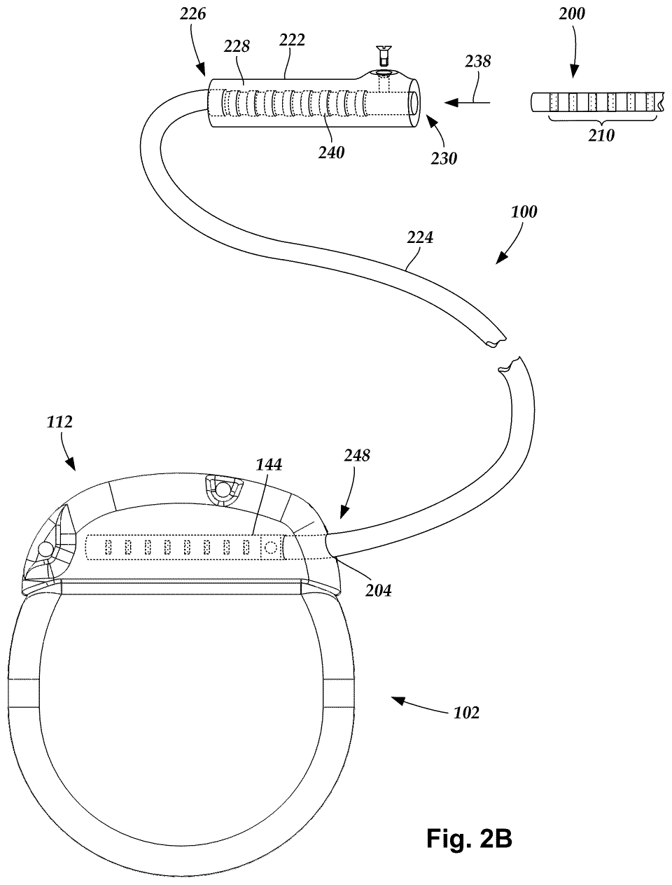

FIG. 1A illustrates schematically one embodiment of an electrical stimulation system 100. The electrical stimulation system includes a control module (e.g., a stimulator or pulse generator, such as an implantable pulse generator (IPG) or external trial stimulation (ETS)) 102 and a cuff lead 103 coupleable to the control module 102. In at least some embodiments, the cuff lead 103 has a distal end portion 226, a central portion 227, and a proximal end portion 248. In at least some embodiments, the cuff lead 103 includes one or more lead bodies 106. In at least some embodiments, the cuff lead 103 includes, at the distal end portion 226 of cuff lead 103, a cuff body 150 and an array 133 of electrodes, such as the electrode 134. In at least some embodiments, the cuff lead 103 further includes one or more lead bodies 106 and an array of terminals (e.g., 210 in FIG. 2A-2B) disposed within the cuff body 150 attached to the one or more lead bodies 106.

In at least some embodiments, the lead body 106 includes an attachment portion 162 at the distal end portion 226 of the lead body 106 for direct attachment to the cuff body 150. In at least some embodiments, the lead body 106 is isodiametric along at least a portion (or all) of the longitudinal length of the lead body 106. FIG. 1A illustrates one cuff lead 103 coupled to a control module 102. Other embodiments may include two, three, four, or more leads 103 coupled to the control module 102. In yet other embodiments, a cuff lead 103 may be coupled to multiple control modules 102. For example, a lead with 64 electrodes may be coupled to two control modules 102 that are capable of handling 32 electrodes each.

The cuff lead 103 can be coupled to the control module 102 in any suitable manner. In at least some embodiments, the cuff lead 103 couples directly to the control module 102. In at least some other embodiments, the cuff lead 103 couples to the control module 102 via one or more intermediate devices (e.g., 200 in FIGS. 2A-2B). For example, in at least some embodiments one or more lead extensions 224 (see e.g., FIG. 2B) can be disposed between the cuff lead 103 and the control module 102 to extend the distance between the cuff lead 103 and the control module 102. Other intermediate devices may be used in addition to, or in lieu of, one or more lead extensions including, for example, a splitter, an adaptor, a device with multiplexing capability (as described, for example, in U.S. Pat. No. 10,350,413, which is incorporated by reference in its entirety) or the like or combinations thereof. It will be understood that, in the case where the electrical stimulation system 100 includes multiple elongated devices disposed between the cuff lead 103 and the control module 102, the intermediate devices may be configured into any suitable arrangement.

The control module 102 typically includes a connector housing 112 and a sealed electronics housing 114. Stimulation circuitry 110 and an optional power source 120 are disposed in the electronics housing 114. A control module connector 144 is disposed in the connector housing 112. The control module connector 144 is configured and arranged to make an electrical connection between the cuff lead 103 and the stimulation circuitry 110 of the control module 102. In at least some embodiments, the control module connector 144 has one or more ports, such as port 191 and port 192.

The electrical stimulation system or components of the electrical stimulation system, including the lead body 106 and the control module 102, are typically implanted into the body of a patient. The electrical stimulation system can be used for a variety of applications including, but not limited to, brain stimulation, neural stimulation, spinal cord stimulation, muscle stimulation, and the like.

The lead body 106 can be made of, for example, a non-conductive, biocompatible material such as, for example, silicone, polyurethane, polyetheretherketone (“PEEK”), epoxy, and the like or combinations thereof. The lead body 106 may be formed in the desired shape by any process including, for example, molding (including injection molding), casting, and the like. The non-conductive material typically extends from the distal end portion of the lead body 106 to the proximal end portion of the lead body 106.

Terminals (e.g., 210 in FIGS. 2A-2B) are typically disposed along the proximal end portion of the lead body 106 of the electrical stimulation system 100 (as well as any splitters, lead extensions, adaptors, or the like) for electrical connection to corresponding connector contacts (e.g., 214 and 240 in FIG. 2B). The connector contacts are disposed in connectors (e.g., 144 in FIGS. 1-2B; and 222 in FIG. 2B) which, in turn, are disposed on, for example, the control module 102 (or a lead extension, a splitter, an adaptor, or the like). Electrically conductive wires 160, cables, or the like (only one of which is shown in FIG. 1A) extend from the terminals to the electrodes 134. Typically, one or more electrodes 134 are electrically coupled to each terminal. In at least some embodiments, each terminal is only connected to one electrode 134.

The electrically conductive wires (“conductors”) 160 (only one of which is illustrated in FIG. 1A for clarity) may be embedded in the non-conductive material of the lead body 106 or can be disposed in one or more lumens (not shown) extending along the lead body 106. In at least some embodiments, the conductors 160 are electrically coupled to the electrodes 134 and extending along the lead body 106. In at least some embodiments, there is an individual lumen for each conductor. In other embodiments, two or more conductors extend through a lumen. There may also be one or more lumens (not shown) that open at, or near, the proximal end portion of the lead body 106, for example, for inserting a stylet to facilitate placement of the lead body 106 within a body of a patient. Additionally, there may be one or more lumens (not shown) that open at, or near, the distal end portion of the lead body 106, for example, for infusion of drugs or medication into the site of implantation of the lead body 106. In at least one embodiment, the one or more lumens are flushed continually, or on a regular basis, with saline, epidural fluid, or the like. In at least some embodiments, the one or more lumens are permanently or removably sealable at the distal end portion.

The conductors 160 (only one of which is illustrated in FIG. 1A for clarity) from within the lead body 106 are received in the attachment portion 162, which in turn is attached to the cuff body 150 such that each conductor passes through the attachment portion 162 for a direct electrical connection with one of the electrodes 134 (e.g., one conductor is electrically connected with one electrode and so on). The attachment portion 162 may be attached in a variety of ways such as, but not limited to, molding or adhering the attachment portion 162 to the cuff body 150. In other embodiments, the conductors 160 from within the lead body 106 are electrically coupled to the electrodes 134 using jumper, intermediate or transition conductors from the lead body 106 to the electrodes 134.

The attachment portion 162 can be offset from the cuff body 150, as illustrated in FIG. 1A, or in-line with the cuff or in any other suitable arrangement. Examples of cuff leads 103 can be found at U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0370793; and 2022/0395690; and U.S. Provisional Patent Application Ser. No. 63/522,367, all of which are incorporated herein by reference in their entireties.

In at least some embodiments, the proximal end portion 248 of the cuff lead 103 couples, directly or through one or more intermediary devices, into the control module 102. For instance, In at least some embodiments, the proximal end portion 248 of the cuff lead 103 is inserted into port 191 of the control module 102. In at least some embodiments, another cuff lead is inserted into port 192 of the control module 102.

For example, FIG. 1B is a schematic view of one embodiment of a portion of a second cuff lead 103′. The proximal end portion (not shown) of the second cuff lead 103′ can be received at port 192 of the control module 102. In at least some embodiments, the cuff lead 103′ has a distal end portion 236. In at least some embodiments, the cuff lead 103′ includes a lead body 106′, conductors 160′, and a cuff body 150′. In at least some embodiments, the cuff body 150′ is at the distal end portion 236 of the cuff lead 103′. The cuff body 150′ has an exterior surface and an interior surface.

In at least some embodiments, the cuff lead 103′ also has, at the distal end portion 236 of cuff lead 103′, an array 133′ of electrodes, such as electrode 134′, on the interior surface of the cuff body 150′, and an array of terminals (e.g., 210 in FIG. 2A-2B) disposed within the cuff body 150′ attached to the lead body 106′. In at least some embodiments, the lead body 106′ includes a distal attachment portion that is directly attached to cuff body 150′. In at least some embodiments, the proximal end portion of the cuff lead 103′ couples, directly or through one or more intermediary devices, into the control module 102.

For instance, in at least some embodiments, the proximal end portion of the cuff lead 103′ inserts into a different port (e.g., port 192) of the control module 102 than the port (e.g., port 191) that cuff lead 103 is inserted into. In these embodiments, the port (e.g., port 191) that cuff lead 103 is inserted into electrically couples the control module 102 to the electrodes 134, and the port (e.g., port 192) that cuff lead 103′ is inserted into electrically couples the control module 102 to the electrodes 134′. In at least some embodiments, the control module 102 generates electrical stimulation signals for delivery to the patient through the electrodes 134, 134′ in the cuff leads (e.g., 103 and 103′) that are inserted into the control module 102.

As described in more detail below, the distal end portion 226 of the cuff lead 103 is conjoinable to the distal end portion 236 of the cuff lead 103′ such that, when the distal end portion 226 of the cuff lead 103 is conjoined with the distal end portion 236 of the cuff lead 103′, the cuff body 150 of the cuff lead 103 and the cuff body 150′ of the cuff lead 103′ are conjoined with each other, adjacent to each other, or near each other. In at least some embodiments, the conjoining of the distal end portion 226 of the cuff lead 103 with the distal end portion 236 of the cuff lead 103′ is accomplished with mating features or other conjoining features on the cuff body 150 and the cuff body 150′ as discussed in greater detail below. The conjoining of the distal end portion 226 of the cuff lead 103 with the distal end portion 236 of the cuff lead 103′ may be performed, for example, during manufacture, during, or prior to, surgery before implantation, or at any another suitable time. Together, cuff lead 103 and cuff lead 103′ are one embodiment of a cuff lead arrangement in which the distal end portions of the cuff leads in the cuff lead arrangement are conjoinable.

Two or more cuff leads, such as the cuff lead 103 and the cuff lead 103′, can be conjoined and disposed around a target nerve (for example, a peripheral nerve such as the vagus nerve). In at least some embodiments, the conjoining can result in a relatively high-density or high-conductor-count cuff arrangement to electrically stimulate a peripheral nerve, such as the vagus nerve or any other suitable nerve. In at least some embodiments, each of the cuff bodies has a particular number of cuff electrodes, such as 4 cuff electrodes each, 8 cuff electrodes each, 12 cuff electrodes each, or another suitable number of cuff electrodes each. For instance, In at least some embodiments, the cuff body 150 has 8 cuff electrodes and the cuff body 150′ has 8 cuff electrodes, and the cuff body 150 and the cuff body 150′ are conjoined so that the conjoined cuff arrangement has a total of 16 cuff electrodes. In these embodiments, each of the cuff leads is inserted into a different port so that all 16 cuff electrodes can be used to provide stimulation. In other embodiments, four different 8-cuff-electrode cuff bodies are conjoined and implanted along a peripheral nerve such as the vagus nerve, with the conjoined cuff arrangement having 32 cuff electrodes, with each of the four corresponding cuff leads being inserted into a separate port of the control module 102.

FIG. 1C is a schematic view of one embodiment of the electrical simulation system 100 that includes a splitter. In the embodiment illustrated in FIG. 1C, the electrical stimulation system 100 is shown having a splitter 107 configured and arranged for facilitating coupling of the cuff lead 103 to the control module 102. The splitter 107 includes a splitter connector 108 configured to couple to a proximal end portion of the cuff lead 103, and one or more proximal tails 109a and 109b configured and arranged to couple to the control module 102 (or another splitter, a lead extension, an adaptor, or the like). The splitter 107 and splitter connector 108 may be part of the cuff lead 103 or may be a separate component that attaches to the lead. Proximal tail 109a is inserted into port 191, and proximal tail 109b is inserted into port 192.

In at least some embodiments, two conjoined or conjoinable cuff bodies 150 are attached to the distal end portion of the same cuff lead 103. The cuff lead 103 can include a splitter 107 with two proximal tails 109a, 109b. In at least some embodiments, the conductors of the proximal tail 109a couple to the electrodes of one of the cuff bodies and the conductors of the proximal tail 109b couple to the electrodes of the other one of the cuff bodies.

FIG. 2A is a schematic side view of one embodiment of a proximal end portion of one or more elongated devices 200 configured and arranged for coupling to one embodiment of the control module connector 144. The one or more elongated devices may include, for example, the lead body 106, the lead body 106′, one or more intermediate devices (e.g., the lead extension 224 of FIG. 2B, an adaptor, or the like or combinations thereof), or a combination thereof. FIG. 2A illustrates two elongated devices 200 coupled to the control module 102. These two elongated devices 200 can be two tails as illustrated in FIG. 1C or two different leads such as cuff lead 103 and cuff lead 103′ or any other combination of elongated devices.

The control module connector 144 defines at least one port into which a proximal end portion of the elongated device 200 can be inserted, as shown by directional arrow 212. In FIG. 2A (and in other figures), the connector housing 112 is shown having two ports 204a and 204b. The connector housing 112 can define any suitable number of ports including, for example, one, two, three, four, five, six, seven, eight, or more ports.

The control module connector 144 also includes a plurality of connector contacts, such as connector contact 214, disposed within each port 204a and 204b. When the elongated device 200 is inserted into the ports 204a and 204b, the connector contacts 214 can be aligned with a plurality of terminals 210 disposed along the proximal end portion(s) of the elongated device(s) 200 to electrically couple the control module 102 to the electrodes (134 of FIG. 1) disposed at a distal end portion of the cuff lead 103. Examples of connectors in control modules are found in, for example, U.S. Pat. Nos. 7,244,150 and 8,224,450, which are incorporated by reference in their entireties.

FIG. 2B is a schematic side view of another embodiment of the electrical stimulation system 100. The electrical stimulation system 100 includes a lead extension 224 that is configured and arranged to couple one or more elongated devices 200 (e.g., the lead body 106, the lead body 106′, an adaptor, another lead extension, or the like or combinations thereof) to the control module 102. In FIG. 2B, the lead extension 224 is shown coupled to a single port 204 defined in the control module connector 144. Additionally, the lead extension 224 is shown configured and arranged to couple to a single elongated device 200. In alternate embodiments, the lead extension 224 is configured and arranged to couple to multiple ports 204 defined in the control module connector 144, or to receive multiple elongated devices 200, or both.

A lead extension connector 222 is disposed on the lead extension 224. In FIG. 2B, the lead extension connector 222 is shown disposed at a distal end portion 226 of the lead extension 224. The lead extension connector 222 includes a connector housing 228. The connector housing 228 defines at least one port 230 into which terminals 210 of the elongated device 200 can be inserted, as shown by directional arrow 238. The connector housing 228 also includes a plurality of connector contacts, such as connector contact 240. When the elongated device 200 is inserted into the port 230, the connector contacts 240 disposed in the connector housing 228 can be aligned with the terminals 210 of the elongated device 200 to electrically couple the lead extension 224 to the electrodes (134 of FIG. 1) disposed along the cuff lead (103 in FIG. 1).

In at least some embodiments, the proximal end portion of the lead extension 224 is similarly configured and arranged as a proximal end portion of the cuff lead 103 (or other elongated device 200). The lead extension 224 may include a plurality of electrically conductive wires (not shown) that electrically couple the connector contacts 240 to a proximal end portion 248 of the lead extension 224 that is opposite to the distal end portion 226. In at least some embodiments, the conductive wires disposed in the lead extension 224 can be electrically coupled to a plurality of terminals (not shown) disposed along the proximal end portion 248 of the lead extension 224. In at least some embodiments, the proximal end portion 248 of the lead extension 224 is configured and arranged for insertion into a connector disposed in another lead extension (or another intermediate device). In other embodiments (and as shown in FIG. 2B), the proximal end portion 248 of the lead extension 224 is configured and arranged for insertion into the control module connector 144.

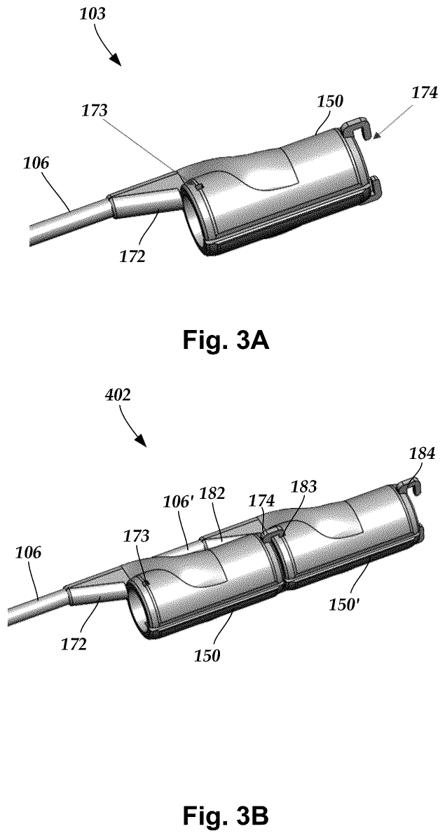

FIG. 3A is a schematic view of one embodiment of a distal end portion 226 of a cuff lead 103 that is conjoinable with the distal end portion of another cuff lead. The cuff body 150 includes: a strain relief 172 that extends over the portion of the lead body 106 that is coupled to the cuff body 150 to reduce strain along this connection, at least one female connection element (for example, the slot 173), and at least one male connection element (for example, the connection finger 174 or plug). In at least some other embodiments, the cuff body 150 may have only one or more female connection elements or one or more male connection elements.

In at least some embodiments, the cuff body 150 is dimensioned and shaped to be disposed at least partially around a stimulation target, for example, a peripheral nerve, such as the vagus nerve, or other suitable tissue, upon implantation of the cuff lead 103. In at least some embodiments, when the cuff lead 103 is implanted, the electrodes 134 (FIG. 1A) are in contact with the stimulation target. During operation of the cuff lead 103, electrical stimulation is provided to the stimulation target via the electrodes 134.

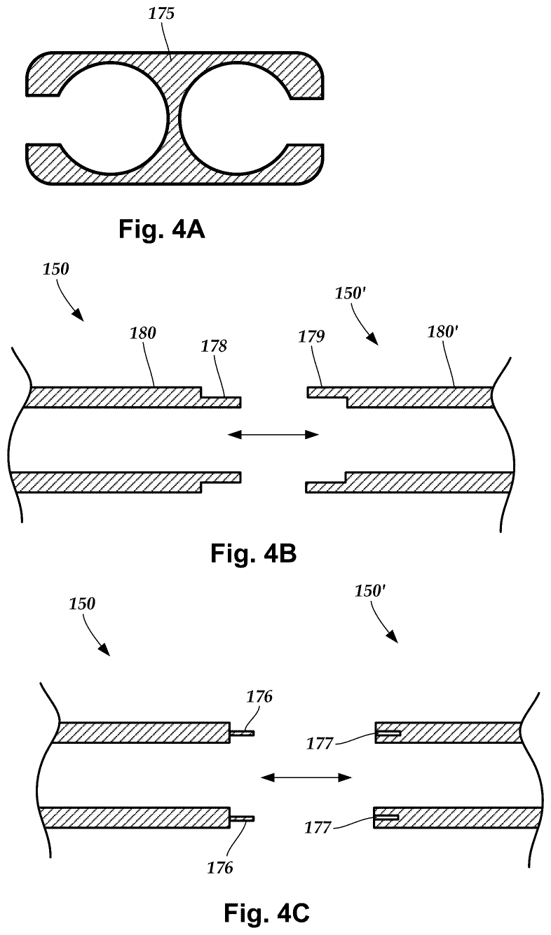

In at least some embodiments, the slot 173 and the connection finger 174 are mating features for conjoining the cuff body 150 to another cuff body 150′. FIG. 3B is a schematic view of a cuff lead arrangement 402 that includes a first cuff body 150, a second cuff body 150′, a first lead body 106, and a second lead body 106′. The second cuff body 150′ includes a strain relief 182, at least one female connection element (for example, the slot 183), and at least one male connection element (for example, the connection finger 184 or plug). In the embodiment illustrated in FIG. 4B, the cuff body 150 is conjoined with the cuff body 150′ by the connection finger 174 of the cuff body 150 being inserted into (or receive by) the slot 183 of the cuff body 150′. In at least some embodiments, the second cuff body 150′ may only include one or more female connection elements or one or more male connection elements. It will be understood that the positions of the male connection elements and the female connection elements on the cuff bodies 150, 150′ of FIGS. 3A and 3B can be switched with one another.

In at least some embodiments, the first cuff body 150 may only include one or more male connection elements and the second cuff body 150′ may only include one or more female connection elements configured to receive the male connection element(s) of the first cuff body. In at least some embodiments, the second cuff body 150′ may only include one or more male connection elements and the first cuff body 150 may only include one or more female connection elements configured to receive the male connection element(s) of the second cuff body.

In at least some embodiments, the conjoined cuff bodies 150 and 150′ combine to provide a cuff lead arrangement with more electrodes than present on the cuff body 150 or the cuff body 150′ alone. In at least some embodiments, the conjoined cuff bodies provide a high-density electrode arrangement with greater electrode density than the cuff body 150 or the cuff body 150′ alone.

FIG. 3A illustrates two connection fingers 174 on the cuff body 150. It will be recognized that a cuff body can have any suitable number of male connection elements (for example, 1, 2, 3, 4, 5, 6, or more male connection elements) or any suitable number of female connection elements (for example, 1, 2, 3, 4, 5, 6, or more female connection elements) or any suitable combination and number of male and female connection elements (for example, 1, 2, 3, 4, 5, 6, 7, 8, 9, 10, 11, 12 or more connection elements).

FIGS. 3A and 3B illustrate cuff bodies 150, 150′ with male connection elements on one end portion of the cuff body and female connection elements on another end of the cuff body. In at least some other embodiments, an arrangement of male and female connection elements (for example, an arrangement with alternative male and female connection elements) can be provided on the same end of the cuff body. The other cuff body can have a complementary arrangement of male and female connection elements on the conjoinable end of that cuff body.

In FIG. 3B, the cuff bodies 150, 150′ are conjoined with the lead bodies 106, 106′ extending from the same circumferential or rotational position of the cuff lead arrangement. In other embodiments, the cuff bodies 150, 150′ can be rotationally offset (e.g., the cuff body 150′ rotated by 90, 180, or 270 degrees (or any other suitable number of degrees) relative to the cuff body 150) to that the lead bodies 106, 106′ extend form different circumferential or rotational positions.

In at least some embodiments, as shown in FIG. 3B, conjoining is accomplished by directly conjoining one cuff with another cuff, such as by using mating features on the cuffs. In other embodiments, conjoining is accomplished, at least in part, with a mating feature on the strain reliefs (e.g., strain reliefs 172 and 182 of FIG. 3B). For instance, in at least some embodiments, the strain reliefs of the cuff leads have complementary male connection element(s) and female connection element(s). In at least some embodiments, conjoining is accomplished based on features on the lead body rather than or in addition to features on the cuff body.

It will be recognized that additional cuff leads can be conjoined to the cuff lead arrangement illustrated in FIG. 3B. For example, a cuff lead arrangement can include two, three, four, or more conjoined cuff leads.

FIGS. 4A to 4C are schematic views of embodiments of other conjoining elements that can be used to conjoin cuff leads. For example, FIG. 4A illustrates a lead clip 175 that can be used to clip together the lead bodies 106, 106′ (or the strain reliefs 172, 182) of the two cuff leads 103, 103′. Preferably, the lead clip 175 is positioned near (for example, within 1, 2, 5, or 10 mm of) one or both of the cuff bodies 150, 150′.

FIG. 4B illustrates another embodiment of a cuff lead arrangement with end portions 177, 179 of the cuff bodies 150, 150′ that conjoin by sliding the end portion 178 (i.e., a male connection element) of cuff body 150 into the end portion 179 (i.e., a female connection element) of cuff body 150′. (It will be understood that the male and female connection elements in FIG. 4B can be switched between cuff bodies 150, 150′.) The end portion 178 of the cuff body 150 has a reduced outer diameter relative to the central portion 180 of the cuff body. The end portion 179 of the cuff lead 103′ has a larger inner diameter relative to the central portion 180′ of the cuff body. In at least some embodiments, adhesive can be provided between the end portions 178, 179 to facilitate coupling of the two cuff bodies 150, 150′.

FIG. 4C illustrates an embodiment of a cuff lead arrangement utilizing pins 176 (male connecting elements) and slots 177 (female connecting elements) for coupling the two cuff bodies 150, 150′ together. Similar to the embodiment of FIG. 3B, multiple pins 176 and multiple slots 177 can be used and the pins and slots may be distributed in any suitable arrangement between the two cuff bodies 150, 150′. In at least some embodiments, all of the pins 176 are on one cuff body and all of the slots 177 are on another cuff body. In at least some embodiments, each cuff body 150, 150′ includes at least one pin 176 and at least one slot 177. In at least some of these embodiments, the pins 176 and slots 177 alternate on the cuff body 150, 150′. Any number of pins 176 can be used including, but not limited to, one, two, three, four, five, six, or more pins. There are at least as many slots 177 as there are pins 176. In at least some embodiments, the material of the cuff body is sufficiently soft that the slots 177 are formed as the pins 176 are pressed into the cuff body.

Any other suitable arrangement can be used to conjoin cuff leads or cuff bodies together. In at least some embodiments, other mechanical male-female fastening or connection is used to conjoin the cuff leads or cuff bodies including, for example, bolt-and-hole, nut-and-hole, screw-and-hole, thread-and-elbow, or the like or any other suitable male-female connection arrangement. Other embodiments use genderless connectors or fasteners, such as the lead clip of FIG. 4A. Other examples include overlapping eyelets (which extend from the cuff bodies 150, 150′ or lead bodies 106, 106′ near the cuff bodies) that are sutured or otherwise coupled together, a tongue-and-groove arrangement, a sliding arrangement, or the like.

In at least some embodiments, the conjoining of cuff leads is enhanced by the application of adhesive on one or both of the connection elements of any of the embodiments described above. In at least some embodiments, the conjoining of the connection elements is reversible so that the cuff leads or cuff bodies can be separated.

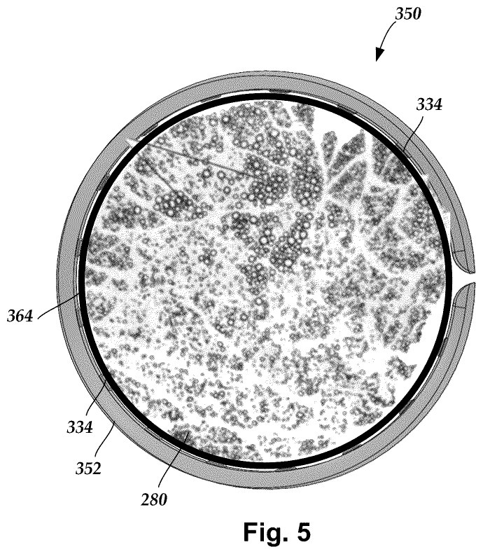

FIG. 5 illustrates a cross-section of a cuff body 350 disposed around a nerve 280 with the electrodes 334 arranged around the circumference of the cuff body and nerve. In at least some embodiments, an interior surface of the cuff body 350 is coated with a cushioning layer 364 to act as a cushion to reduce damage to the nerve. Examples of materials for the cushioning layer 364 include, but are not limited to, paraffin, a combination of isotonic saline and artificial cerebrospinal fluid, polyethylene glycol, or the like or any combination thereof. In at least some embodiments, the cushioning layer 364 is made of a material that permits flow of current from the electrodes 334 to the nerve through the cushioning layer.

When the cuff bodies are conjoined, any suitable arrangement of electrodes or other stimulation emitters can be used on the cuff bodies. Non-limiting examples of electrode arrangements can be found in U.S. Pat. Nos. 7,596,414; 7,974,706; 8,423,157; 10,485,969; 10,493,269; 10,709,888; and 10,814,127; U.S. Patent Application Publications Nos. 2017/0333692; 2018/0154156; 2022/0370793; and 2022/0395690; and U.S. Provisional Patent Application Ser. No. 63/522,367, all of which are incorporated herein by reference in their entireties.

The arrangement of electrodes on the different cuff bodies can be the same or different. In at least some embodiments, the electrodes on each of the conjoined cuff bodies is aligned with the electrodes on one or more other conjoined cuff bodies. In at least some embodiments, the electrodes on one cuff body are offset (e.g., rotationally or circumstantially offset) relative to the electrodes on a conjoined cuff body. In at least some embodiments, the electrodes on one cuff body are clockwise rotated relative to the electrodes on another conjoined cuff body. Any suitable pattern of rotational offset or alignment of the electrodes relative to each other can be used. In at least some embodiments, the electrodes on each of the conjoined cuff bodies are arranged together in a pattern that enhances stimulation selectivity by providing electrodes at different axial and rotational positions around the lead.

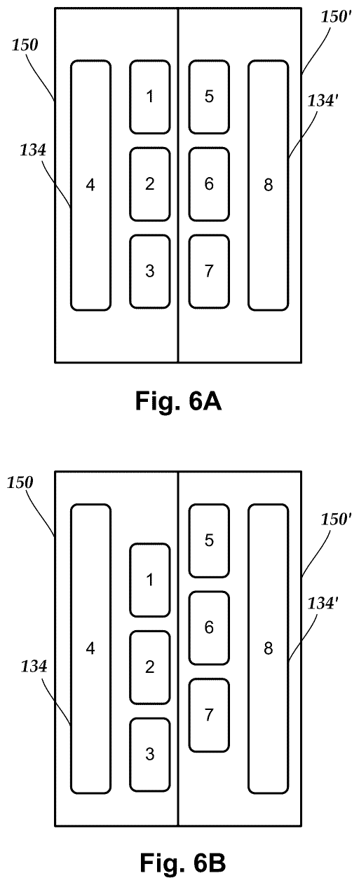

FIGS. 6A and 6B illustrate examples of two cuff bodies 150, 150′ with different electrode arrangements. One cuff body 150 includes electrodes 1 to 4 and the other cuff body includes electrodes 5 to 8. It will be understood that these electrode arrangements are presented for illustration purposes and that any other suitable electrode arrangements and number of electrodes can be used. FIGS. 6A and 6B each illustrate a plan view of with the cuff bodies 150, 150′ unwrapped to illustrate the arrangement of the electrodes. In the illustrated embodiments of FIG. 6A and FIG. 6B, the vertical edges become the circumferential edges when the cuff bodies are wrapped.

In these embodiments, the four vertical rows of electrodes provide four different axial or longitudinal positions for stimulation. In the arrangement illustrated in FIG. 6A, the two vertical rows of three electrodes provide three different circumferential or rotational stimulation positions at each of those two axial or longitudinal positions. In the arrangement illustrated in FIG. 6B, the two adjacent vertical rows of the two cuff bodies are offset or staggered relative to each other. In the offset or staggered embodiment illustrated in FIG. 6B, there are six different circumferential or rotational stimulation positions distributed over the two axial or longitudinal positions.

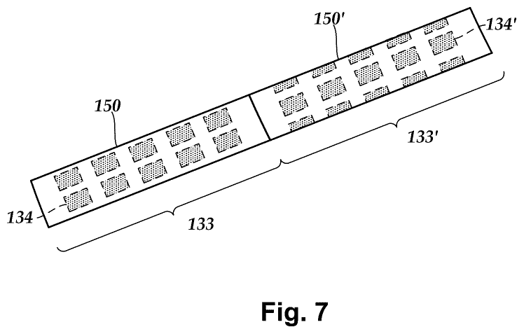

As discussed above, in at least some embodiments, the electrodes on one cuff body are rotated relative to the electrodes on another conjoined cuff body. As an example, FIG. 7 illustrates one embodiment of conjoined cuff bodies in which the electrodes on one cuff body 150′ are clockwise rotated relative to the electrodes on another cuff body 150. The cuff body 150′ is conjoined with the cuff body 150. The cuff body 150 includes the array of electrodes 133 including the electrode 134. The cuff body 150′ includes the array of electrodes 133′ including the electrode 134′. As illustrated in FIG. 7, the electrodes in array of electrodes 133′ of the cuff body 150′ are clockwise rotated relative to the electrodes in the array of electrodes 133 in the cuff body 150.

Any suitable method can be used for manufacturing the cuff leads. For example, conductors can be welded to the electrodes which are dispersed in a non-conducting (for example, silicone) carrier. A non-conductive layer may cover the conductors and this construct can be formed in the shape of a cuff. The cuff body can be formed of any suitable biocompatible and biostable non-conductive material including, but not limited to, polymer materials such as silicone, polyurethane, polyetheretherketone (“PEEK”), epoxy, or the like. In at least some embodiments, the cuff bodies can be formed using any suitable technique including, but not limited to, molding, casting, formed in a sheet and then shaped using adhesive as a binder, formed flat and shaped using heat, formed flat and attached to a cuff-shaped scaffold, pressed, or extruded into the cuff shape, assembled by adhering sheets together, or the like or any combination thereof. In at least some embodiments, the electrodes can be attached to the cuff bodies using any suitable technique including, but not limited to, attaching with adhesive, molding (for example, insert molding) into the cuff body, using heat to adhere the electrodes to the cuff body, heating and pressing the electrodes into the cuff body, depositing electrode material on the cuff body, and using photolithography and etching, or the like or any combination thereof.

In various embodiments, other techniques are used to form the electrodes including, but not limited to, photolithography, e-beam lithography, electrodeposition, or sputtering of the electrodes on a substrate or laser cutting or ablation of electrode material disposed on/in, or disposable on/in, a non-conductive carrier. In at least some embodiments, each cuff body can have a circular, oval, or any other suitable cross-sectional shape and, in at least some embodiments, may be sufficiently flexible to alter the cross-sectional shape to accommodate the nerve. In at least some embodiments, the electrodes can be molded with the cuff bodies or formed by techniques such as etching or ablation of conductive layers, films, or the like. In at least some embodiments, each of the cuff bodies has an inner diameter (which can correspond to the largest diameter of a non-circular cuff body) in a range of 0.5 to 5.5 mm or in a range of 1 to 3 mm. In at least some embodiments, each of the cuff bodies has a length of at least 5, 10, 20, 30, 40, or 50 mm or more. In at least some embodiments, each cuff is configured to fit around a portion of the vagus, splanchnic, hepatic, hypogastric, or other peripheral nerves or other tissue.

In at least some embodiments, the electrodes are rectangular or rectangular with rounded corners. In various embodiments, any other suitable shape can be used for the electrodes including, but not limited to, oblong, oval, modified rectangular with one or more sides (or portions of sides) that are curved, or the like or any combination thereof. In at least some embodiments, the electrodes can be formed using any conductive, biocompatible material. Examples of suitable materials include metals, alloys, conductive polymers, conductive carbon, and the like, as well as combinations thereof. In at least some embodiments, one or more of the electrodes are formed from one or more of: platinum, platinum alloys such as platinum iridium, palladium alloys such as palladium rhodium, titanium, titanium alloys, nickel alloys, cobalt alloys, nickel/cobalt alloys, stainless steels, tantalum, conductive carbon, conductive plastics, epoxy, or other adhesive filled with metallic powder, Nitinol™, or the like or any combination thereof.

In at least some embodiments, the electrodes have a contact surface that is flush or slightly protruding (for example, no more than 200, 100, or 50 μm) from the cuff body which, at least in some circumstances, may reduce or eliminate physical pressure on the nerve. It will be recognized that the electrodes can be used to provide electrical stimulation or to sense electrical signals from tissue or any combination thereof.

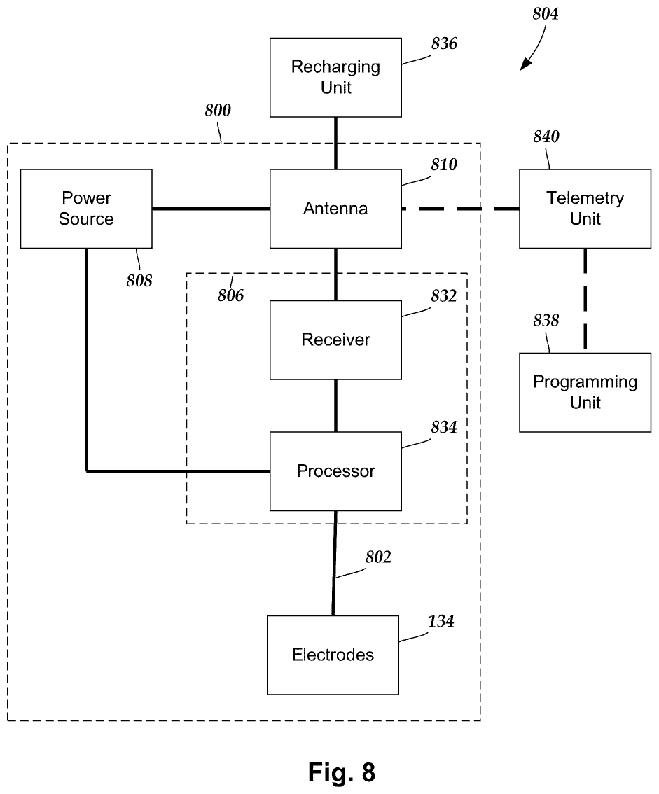

FIG. 8 is a schematic overview of one embodiment of components of an electrical stimulation arrangement 804 that includes an electrical stimulation system 800 with a lead 802, stimulation circuitry 806, a power source 808, and an antenna 810. The electrical stimulation system can be, for example, any of the electrical stimulation systems described above. It will be understood that the electrical stimulation arrangement can include more, fewer, or different components and can have a variety of different configurations including those configurations disclosed in the stimulator references cited herein.

If the power source 808 is a rechargeable battery or chargeable capacitor, the power source may be recharged/charged using the antenna 810, if desired. Power can be provided for recharging/charging by inductively coupling the power source 808 through the antenna 810 to a recharging unit 836 external to the user. Examples of such arrangements can be found in the references identified above.

In at least some embodiments, electrical current is emitted by the electrodes (such as electrodes 134 in FIG. 1A) on the lead 802 to stimulate nerve fibers, muscle fibers, or other body tissues near the electrical stimulation system. In this way, in these embodiments, electrical current is applied to the patient via the electrodes. In at least some embodiments, electrical current is applied to the patient via the electrodes in one cuff lead, and electrical current is applied to the patient via electrodes in another cuff link that is conjoined with the cuff link. The stimulation circuitry 806 can include, among other components, a processor 834 and a receiver 832. The processor 834 is included to control the timing and electrical characteristics of the electrical stimulation system. For example, the processor 834 can, if desired, control one or more of the timing, frequency, strength, duration, and waveform of the pulses. In addition, the processor 834 can select which electrodes can be used to provide stimulation, if desired (see, for example, U.S. Pat. No. 8,412,345, which is incorporated herein by reference in its entirety). In at least some embodiments, the processor 834 selects which electrode(s) are cathodes and which electrode(s) are anodes. In at least some embodiments, the processor 834 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 838 that, for example, allows modification of pulse characteristics. In the illustrated embodiment, the processor 834 is coupled to a receiver 832 which, in turn, is coupled to the antenna 810. This allows the processor 834 to receive instructions from an external source to, for example, direct the pulse characteristics and the selection of electrodes, if desired.

In at least some embodiments, the antenna 810 is capable of receiving signals (e.g., RF signals) from an external telemetry unit 840 that is programmed by the programming unit 838. The programming unit 838 can be external to, or part of, the telemetry unit 840. The telemetry unit 840 can be a device that is worn on the skin of the user or can be carried by the user and can have a form similar to a pager, cellular phone, or remote control, if desired. As another alternative, the telemetry unit 840 may not be worn or carried by the user but may only be available at a home station or at a clinician's office. The programming unit 838 can be any unit that can provide information to the telemetry unit 840 for transmission to the electrical stimulation system 800. The programming unit 838 can be part of the telemetry unit 840 or can provide signals or information to the telemetry unit 840 via a wireless or wired connection. One example of a suitable programming unit is a computer operated by the user or clinician to send signals to the telemetry unit 840.

The signals sent to the processor 834 via the antenna 810 and the receiver 832 can be used to modify or otherwise direct the operation of the electrical stimulation system 800. For example, the signals may be used to modify the pulses of the electrical stimulation system such as modifying one or more of pulse duration, pulse frequency, pulse waveform, and pulse strength. The signals may also direct the electrical stimulation system 800 to cease operation, to start operation, to start charging the battery, or to stop charging the battery.

Optionally, the electrical stimulation system 800 may include a transmitter (not shown) coupled to the processor 834 and the antenna 810 for transmitting signals back to the telemetry unit 840 or another unit capable of receiving the signals. For example, the electrical stimulation system 800 may transmit signals indicating whether the electrical stimulation system 800 is operating properly or not or indicating when the battery needs to be charged or the level of charge remaining in the battery. The processor 834 may also be capable of transmitting information about the pulse characteristics so that a user or clinician can determine or verify the characteristics.

The above specification and examples provide a description of the arrangement and use of the invention. Since many embodiments of the invention can be made without departing from the spirit and scope of the invention, the invention also resides in the claims hereinafter appended.

What is claimed as new and desired to be protected is:

1. A cuff lead arrangement for electrical stimulation of a nerve, the cuff lead arrangement comprising:
a first cuff lead, comprising
a first cuff body configured to be disposed around at least a portion of the nerve, the first cuff body comprising an exterior surface, an interior surface, and at least one first male connection element,
a plurality of first electrodes disposed on the interior surface of the first cuff body,
a first lead body coupled to the first cuff body, and
a plurality of first conductors electrically coupled to the first electrodes and extending along the first lead body; and
a second cuff lead, comprising
a second cuff body configured to be disposed around at least a portion of the nerve, the second cuff body comprising an exterior surface, an interior surface, and at least one second female connection element configured to receive the at least one first male connection element to conjoin the first cuff body to the second cuff body,
a plurality of second electrodes disposed on the interior surface of the second cuff body,
a second lead body coupled to the second cuff body, and
a plurality of second conductors electrically coupled to the second electrodes and extending along the second lead body.

2. The cuff lead arrangement of claim 1, wherein each of the at least one first male connection element comprises a connection finger and each of the at least one second female connection element comprises a slot configured to receive a portion of the connection finger to conjoin the first cuff body to the second cuff body.

3. The cuff lead arrangement of claim 1, wherein the at least one first male connection element comprises a first end portion of the first cuff body having a reduced outer diameter relative to a first central portion of the first cuff body and the at least one second female connection element comprises a second end portion of the second cuff body having a larger inner diameter relative to a second central portion of the second cuff body, wherein the second end portion of the second cuff body is configured to receive therein the first end portion of the first cuff body to conjoin the first cuff body to the second cuff body.

4. The cuff lead arrangement of claim 1, wherein the first cuff body further comprises a first strain relief extending along a portion of the first lead body wherein the first lead body is attached to the first cuff body, wherein the at least one male connection element is part of the first strain relief.

5. The cuff lead arrangement of claim 1, wherein each of the at least one first male connection element comprises a pin and each of the at least one second female connection element comprises a slot configured to receive the pin to conjoin the first cuff body to the second cuff body.

6. The cuff lead arrangement of claim 1, wherein the at least one first male element and the at least one second female connection element comprise at least one of: a bolt-and-hole arrangement, a nut-and-hole arrangement, a screw-and-hole arrangement, or a thread-and-elbow arrangement.

7. The cuff lead arrangement of claim 1, wherein the first cuff body further comprises at least one first female connection element and the second cuff body further comprises at least one second male connection element configured to receive the at least one first female connection element to conjoin the first cuff body to the second cuff body.

8. The cuff lead arrangement of claim 1, wherein the first cuff body comprises a plurality of the first male connection elements and the second cuff body comprises a plurality of the second female connection elements configured to receive the first male connection elements to conjoin the first cuff body to the second cuff body.

9. The cuff lead arrangement of claim 1, wherein the second cuff body further comprises at least one second male connection element, the cuff lead arrangement further comprising
a third cuff lead, comprising
a third cuff body configured to be disposed around at least a portion of the nerve, the third cuff body comprising an exterior surface, an interior surface, and at least one third female connection element configured to receive the at least one second male connection element to conjoin the second cuff body to the third cuff body,
a plurality of third electrodes disposed on the interior surface of the third cuff body,
a third lead body coupled to the third cuff body, and
a plurality of third conductors electrically coupled to the third electrodes and extending along the third lead body.

10. The cuff lead arrangement of claim 1, wherein the first electrodes are disposed in a first arrangement on the interior surface of the first cuff lead and the second electrodes are disposed in the first arrangement on the interior surface of the second cuff lead.

11. The cuff lead arrangement of claim 10, wherein, when conjoined, the second electrodes in the first arrangement are rotationally offset from the first electrodes in the first arrangement.

12. An electrical stimulation system, comprising:
the cuff lead arrangement of claim 1; and
a control module configured to receive a portion of the first lead body and to electrically couple to the first plurality of electrodes and to receive a portion of the second lead body and to electrically couple to the second plurality of electrodes, wherein the control module is configured to generate electrical stimulation signals for delivery through at least one of the first or second electrodes.

13. A method of electrically stimulating a patient using the electrical stimulation system of claim 12, the method comprising:
providing the cuff lead arrangement implanted within the patient;
generating electrical stimulation signals by the control module; and
delivering the electrical stimulation signals from the control module to the patient using at least one of the first or second electrodes.

14. A cuff lead arrangement for electrical stimulation of a nerve, the cuff lead arrangement comprising:
a first cuff lead, comprising
a first cuff body configured to be disposed around at least a portion of the nerve,
a plurality of first electrodes disposed on an interior surface of the first cuff body,
a first lead body comprising a first distal attachment portion directly attached to the first cuff body,
a plurality of first conductors electrically coupled to the first electrodes and extending along the first lead body, and
at least one first connection element coupled to the first cuff body or the first distal attachment portion of the first lead body; and
a second cuff lead, comprising
a second cuff body configured to be disposed around at least a portion of the nerve,
a plurality of second electrodes disposed on an interior surface of the second cuff body,
a second lead body comprising a second distal attachment portion directly attached to the second cuff body,
a plurality of second conductors electrically coupled to the second electrodes and extending along the second lead body, and
at least one second connection element coupled to the second cuff body or the second distal attachment portion of the second lead body, wherein the at least one second connection element is configured to be attached to the at least one first connection element to conjoin the first cuff lead to the second cuff lead.

15. The cuff lead arrangement of claim 14, wherein the at least one first connection element and the at least one second connection element comprise at least one of: a tongue-and-groove arrangement, a screw arrangement, a bolt arrangement, a thread-and-elbow arrangement, a pin-and-slot arrangement, a connection-finger-and-slot arrangement, or a sliding arrangement.

16. The cuff lead arrangement of claim 14, wherein the at least one first connection element comprises a first eyelet coupled to the first cuff body or the first distal attachment portion, the at least one second connection element comprises a second eyelet coupled to the second cuff body or the second distal attachment portion, and the first eyelet and the second eyelet are configured to be sutured together.

17. The cuff lead arrangement of claim 14, wherein the second cuff lead further comprises at least one third connection element coupled to the second cuff body of the second distal attachment portion of the second lead body, the cuff lead arrangement further comprising
a third cuff lead, comprising
a third cuff body configured to be disposed around at least a portion of the nerve,
a plurality of third electrodes disposed on an interior surface of the third cuff body,
a third lead body comprising a third distal attachment portion directly attached to the third cuff body,
a plurality of third conductors electrically coupled to the third electrodes and extending along the third lead body, and
at least one third connection element coupled to the third cuff body or the third distal attachment portion of the third lead body, wherein the at least one third connection element is configured to be attached to the at least one second connection element to conjoin the second cuff lead to the third cuff lead.

18. An electrical stimulation system, comprising:
the cuff lead arrangement of claim 14; and
a control module configured to receive a portion of the first lead body and to electrically couple to the plurality of first electrodes and to receive a portion of the second lead body and to electrically couple to the plurality of second electrodes, wherein the control module is configured to generate electrical stimulation signals for delivery through at least one of the first or second electrodes.

19. A method of electrically stimulating a patient using the electrical stimulation system of claim 14, the method comprising:
providing the cuff lead arrangement implanted within the patient;
generating electrical stimulation signals by the control module; and
delivering the electrical stimulation signals from the control module to the patient using at least one of the first or second electrodes.

20. A cuff lead arrangement for electrical stimulation of a nerve, the cuff lead arrangement comprising:
a first cuff lead, comprising
a first cuff body configured to be disposed around at least a portion of the nerve,
a plurality of first stimulation emitters disposed on the interior surface of the first cuff body,
a first lead body comprising a first distal attachment portion directly attached to the first cuff body,
a plurality of first conductors operatively coupled to the first stimulation emitters and extending along the first lead body, and
at least one first connection element coupled to the first cuff body or the first distal attachment portion of the first lead body; and
a second cuff lead, comprising
a second cuff body configured to be disposed around at least a portion of the nerve,
a plurality of second stimulation emitters disposed on the interior surface of the second cuff body,
a second lead body comprising a second distal attachment portion directly attached to the second cuff body,
a plurality of second conductors operatively coupled to the second stimulation emitters and extending along the second lead body, and
at least one second connection element coupled to the second cuff body or the second distal attachment portion of the second lead body, wherein the at least one second connection element is configured to be attached to the at least one first connection element to conjoin the first cuff lead to the second cuff lead.
